(19) United States
(12) Patent Application Publication
(10) Pub. No.: US 2010/0163021 A1
(43) Pub. Date: Jul. 1, 2010

(54) SYSTEM FOR DELIVERING NEBULIZED CYCLOSPORINE AND METHODS OF TREATMENT

(75) Inventor: Johnny Lai, Seattle, WA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS PHARMA AG, Basel (CH)

(21) Appl. No.: 12/280,120

(22) PCT Filed: Feb. 22, 2007
(86) PCT No.: PCT/US2007/004861

§ 371 Date: Oct. 29, 2008

Related U.S. Application Data
(60) Provisional application No. 60/775,919, filed on Feb. 22, 2006.

(51) Int. Cl. A61M 11/00

(52) U.S. Cl. 128/200.23

(57) ABSTRACT

Systems comprising a pressurized delivery device and a formulation of cyclosporine coupled to an exhalation filter or trap that is capable of preventing cyclosporine from escaping into the local environment are provided. An apparatus for use in the system comprises either an exhalation filter and a pressurized delivery device, wherein the exhalation filter is capable of providing high filter efficiency and maintaining low filter resistance after usage with the formulation, or a trap which provides a means for expired gas to be released into a solvent chamber containing a solvent with a high affinity for cyclosporine. These systems may be used to treat patients with pulmonary disorders, organ transplant patients such as lung transplant patients, and other immune-related disorders.

### CROSS-REFERENCE TO RELATED APPLICATIONS

This application claims the priority benefit of U.S. Provisional Patent Application No. 60/775,919 filed on Feb. 22, 2006. The priority application is hereby incorporated herein by reference in its entirety.

### TECHNICAL FIELD

The invention relates to methods and systems for delivering aerosolized cyclosporine comprising an exhalation filter or trap that minimizes or prevents the escape of cyclosporine particles into the environment upon exhalation of the aerosolized cyclosporine. In general, the system comprises a pressurized delivery device and a trap or filter having a suitable filter efficiency and filter resistance upon usage.

### BACKGROUND ART

Aerosolized cyclosporine delivery systems provide cyclosporine to the lung. Patent Publication No. US 2002/0006901 describes various methods and compositions for using aerosolized cyclosporine, for example, for the prevention of graft rejection in lung transplant recipients. This publication is incorporated herein by reference. Aerosolized cyclosporine may be provided as a nebulized solution with a nebulizer. A filter or trap may be attached to the expiratory part of the nebulizer (“exhalation filter” or “exhalation trap”) to capture particles or material that are exhaled by the user, such as particles that were never inhaled. In a standard delivery device such as a nebulizer, the exhalation filter may have an increased resistance with use, and the filter may get clogged by the exhalation of a carrier or solvent that is present with the cyclosporine, and/or may also fail to capture cyclosporine particles and thus allow the particles to escape into the local environment.

Therefore, there is a need for a delivery system for administration of aerosolized cyclosporine provided as a nebulized solution which relies on a device such as a nebulizer and which comprises a filter that maintains low filter resistance during use, or comprises a solvent trap that captures escaping particles, such that cyclosporine is prevented from entering the environment upon exhalation of the aerosolized cyclosporine.

### SUMMARY OF THE INVENTION

One aspect of the invention is directed to an apparatus for delivering aerosolized cyclosporine comprising an exhalation filter or trap that minimizes or prevents the escape of cyclosporine particles into the environment, comprising a pressurized delivery device coupled to an exhalation filter, or trap wherein the exhalation filter is capable of maintaining a filter efficiency of greater than 90% during usage with a formulation comprising propylene glycol and cyclosporine for aerosolization by the device; and is capable of maintaining an increase in filter resistance between a new filter and after usage of less than 0.1 cmH₂O^{0.5}·min/L.

A further aspect is directed to a system comprising the apparatus described above and a formulation comprising a liquid formulation of cyclosporine for aerosolization by the device. In one approach, the exhalation filter is a hydrophobic high efficiency particulate air (HEPA) filter, such as one that comprises a polypropylene and/or acrylic medium, for example an Isogard HEPA Light filter. Preferably, the pressurized delivery device is a nebulizer. In another approach, the system includes a trap, for example a trap which comprises a solvent with a high affinity for cyclosporine, such that the solvent trap captures escaping cyclosporine particles.

In a preferred embodiment, the formulation is present in an amount less than 10 mL, less than 7 mL, less than 5 mL, less than 3 mL, or less than 2 mL. One preferred formulation contains 62.5 mg/ml of cyclosporine A in a solvent, e.g., propylene glycol or ethanol, such as a formulation containing 325 mg cyclosporine and 5.2 ml of solvent.

Another aspect of the invention is directed to a method of treating a pulmonary disorder comprising administering aerosolized cyclosporine using the system defined above, to a lung of a subject having a pulmonary disorder, such as cystic fibrosis, and in another aspect, wherein the lung is a transplanted lung.

Another embodiment is directed to a method of preventing graft rejection in an organ transplant patient comprising administering to a subject an effective dose of aerosolized cyclosporine using the system described above, such as wherein the organ is a lung. In one embodiment, the formulation is administered directly after lung transplantation.

Another embodiment is directed to a method of capturing exhaled cyclosporine using by passing expired gas through a liquid reservoir or ‘trap’. In this embodiment more than 90% of exhaled cyclosporine is captured by the trap. The trap liquid consists of an organic solvent such as ethanol or propylene glycol capable of capturing a great proportion of cyclosporine in solution.

A further aspect is directed to a method for selecting an exhalation filter for use in a system comprising a nebulizer coupled to the exhalation filter; wherein the nebulizer contains a solution comprising propylene glycol and cyclosporine, comprising a) turning on a compressor and a respirator pump in a breathing apparatus to nebulize the solution; wherein the apparatus comprises a test exhalation filter, the compressor, the respirator pump, an inhalation filter, a trap filter, and a nebulizer containing cyclosporine and propylene glycol wherein the components of the apparatus are coupled to each other to form the breathing apparatus capable of simulating breathing; b) turning off the compressor and the respirator pump after the nebulizer runs dry; c) measuring the test filter efficiency by testing the trap filter to measure the quantity of cyclosporine that passed through the test filter; and d) measuring the filter resistance of the test filter; wherein a filter efficiency of greater than 90%, and a difference in filter resistance between the test filter before the turning on step and after the turning off step of less than 0.1 cmH₂O^{0.5}·min/L, are indicative of an appropriate exhalation filter to be used with said solution.

### BRIEF DESCRIPTION OF THE DRAWINGS

FIG. 1 is the equipment set up for the exhalation filter challenge of Examples 1-2.

### DETAILED DESCRIPTION OF THE INVENTION

In an important aspect of the invention, the exhalation filter interferes with or prevents cyclosporine from entering the environment upon exhalation of the aerosolized cyclosporine. Preferably, the filter efficiency is greater than 90% and more preferably greater than 95%, and more preferably greater than 98%. Most preferably, the filter efficiency is greater than 99%. The filter efficiency is calculated by the following formula:

$$\begin{matrix}Calculated filter efficiency ( \% ) = 100 \% - ( \begin{matrix}\% exhalation aerosol \\ escaped past \\ challange filter\end{matrix} ) \\ = 100 - [ \frac{( \begin{matrix}mg CsA collected \\ on trap filter\end{matrix} )}{132.3 mg} ] \times \\ 100\end{matrix}$$

The most preferred filter for use in the system of the invention is the Iso-Gard HEPA Light Filter, which is a pleated, highly hydrophobic bacterial/viral filter. This filter is classified as a high-efficiency particulate air (HEPA) exhalation filter in HEPA Class 13. Other HEPA filters, such as Class 13 HEPA filters, may also be used.

In another important aspect, the exhalation filter maintains low filter resistance during use. Such maintenance will prevent clogging of the filter by carriers such as propylene glycol. Preferably, the new filter has a resistance specification for comfortable tidal breathing of less than about 0.10 cm H₂O^{0.5}·min/L or less, such as 0.06 cm H₂O^{0.5}·min/L. Preferably, the difference in filter resistance between the new filter and the filter after use should not significantly increase. Preferably, the difference in filter resistance between a new and used filter should be less than 0.1 cm H₂O^{0.5}·min/L, more preferably less than 0.05 cmH₂O^{0.5}·min/L, even more preferably less than 0.03 cmH₂O^{0.5}·min/L, and most preferably less than 0.02 cmH₂O^{0.5}·min/L.

Filter efficiency and filter resistance may be measured in accordance with methods described herein. Thus, filters may be selected that conform to the selected criteria and used in the system and methods of the invention. The Isogard HEPA Light filter (Hudson RCI, Upplands Väsby, Sweden) has approximately 99% efficiency and difference in filter resistance of about 0.02-0.03 cmH₂O^{0.5}·min/L. The medium in the Isogard HEPA light filter is a pleated polypropylene and acrylic fiber medium which is a Technostat T-200 medium (Hollingsworth & Vose Air Filtration Ltd., Cumbria, UK). In addition to polypropylene and/or acrylic, hydrophobic media such as those described in U.S. Pat. No. 5,320,096, which is incorporated herein by reference, may be used such as compressed hydrophobized glass fibers, polysulphone, polycarbonate fibers, or combinations thereof. Other filters having similar efficiency and filter resistance properties are also preferred.

The AeroTech II filter (CIS-US, Bedford Mass.), which is a bacterial/viral filter made from polypropylene, is not preferred as this filter has a low filter efficiency, although the filter resistance measured after use otherwise would be acceptable. In contrast, the Conserve™ Breathing Circuit Filter (Pall Corp., East Hills, N.Y.) has high filter efficiency, but the filter resistance after usage is not acceptable, and thus this filter is also not preferred. The Conserve™ filter comprises a hydrophobic resin bonded to (hydrophilic) inorganic fibers. Although not bound by this theory, it appears that the hydrophilic fibers absorb and retain the propylene glycol carrier, thus increasing filter resistance after usage. Thus, preferably hydrophobic filters are used which do not contain hydrophilic portions.

A breathing simulator may be used in an apparatus that comprises an inhalation filter, a pressurized delivery device, a test filter, a trap filter, and a compressor. The breathing simulator, such as a respirator pump, may be set at various settings. Preferred settings are an respiratory rate of 15 breaths/min., a percent inspiration of 50%, and a cc per stroke which is the tidal volume (Vₜ) which is about 500 mL. A nebulizer containing a liquid formulation of cyclosporine may be used. Once the compressor is turned on, the nebulizer is emptied and then the compressor and pump may be turned off. The resistance of the test filter is measured by connecting it to a breathing simulator wherein the resistance is measured. The trap filter, which will trap the particles that escape from the exhalation filter, may be tested to quantify the filter efficiency. For example, the trap filter may be extracted with ethanol one or more times and tested for the mass of cyclosporine that escaped from the test filter.

A pressurized delivery device or any nebulizer may be used in the system of the invention. Preferably, aerosolized cyclosporine is provided in particle sizes of less than about 5 μm, and preferably less than 3 μm, and more preferably less than 2 μM. For example, commercially available jet nebulizers may deliver aerosolized cyclosporine to a subject. Such jet nebulizers include, but are not limited to, those supplied by AeroTech II (CIS-US, Bedford, Mass.). In addition, for delivery of aerosolized cyclosporine to the lungs of a subject, an oxygen source can be attached to the nebulizer providing a flow rate of, for example, 10 L/min. In general, inhalation may be performed over a 30-40 minute time interval through a mouthpiece during spontaneous respiration.

In one exemplary embodiment, a trap device is connected to the mouthpiece of the inhalation portion of the delivery device such that by means of actuation of a two-way valve, expiration by the patient closes the inlet valve and passes the expired air into the trap receptacle. The trap receptacle consists of a chamber containing a solvent with a high affinity for cyclosporine. Exemplary solvents, include, but are not limited to propylene glycol and ethanol. The trap receptacle will typically contain the same solvent which is found in the cyclosporine formulation that is being administered. In this embodiment, the expired gas is released through the solvent by a simple bubbling process such that any cyclosporine contained within the gas dissolves in the solvent. The gas that bubbles out of the solvent is evacuated from the trap receptacle to the environment. This embodiment provides a means to significantly decrease the amount of immunosuppressant released into the air surrounding the patient, and will result in less restriction of patient care giver presence.

The carrier solvent used in the system of the invention is propylene glycol, ethanol or another solvent or lipid.

Cyclosporine is a potent immunosuppressive agent that in animals prolongs survival of allogeneic transplants involving skin, kidney, liver, heart, kidney, pancreas, bone marrow, small intestine, and lung. Cyclosporine has been demonstrated to suppress some humoral immunity and to a greater extent, cell-mediated reactions such as allograft rejection, delayed hypersensitivity, experimental allergic encephalomyelitis, Freund's adjuvant arthritis, and graft vs. host disease in many animal species for a variety of organs.

### PULMINIQ™

One preferred cyclosporine formulation is PULMINIQ™ (manufactured by Novartis Pharma Stein A.G., 4332 Stein, Switzerland. PULMINIQ™ is a sterile, clear, colorless, preservative-free solution of cyclosporine in propylene glycol developed specifically for administration by oral inhalation. The active principle of PULMINIQ™ is a cyclic polypeptide immunosuppressant agent consisting of 11 amino acids. It is produced as a metabolite by the fungus species Beauveria nivea. The molecular formula of cyclosporine used in PULMINIQ™ is C₆₂H₁₁₁N₁₁O₁₂ and the molecular weight is 1202.63. The chemical name for cyclosporine is [R—[R*,R*-(E)]]-cyclic(L-alanyl-D-alanyl-N-methyl-L-leucyl-N-methyl-L-leucyl-N-methyl-L-valyl-3-hydroxy-N,4-dimethyl-L-2-amino-6-octenoyl-L-α-amino-butyryl-N-methylglycyl-N-methyl-L-leucyl-L-valyl-N-methyl-L-leucyl). The carrier used in the system of the invention when the cyclosporine formulation is PULMINIQ™ is propylene glycol.

The chemical structure of cyclosporine found in PULMINIQ™ (also known as cyclosporine A) is:

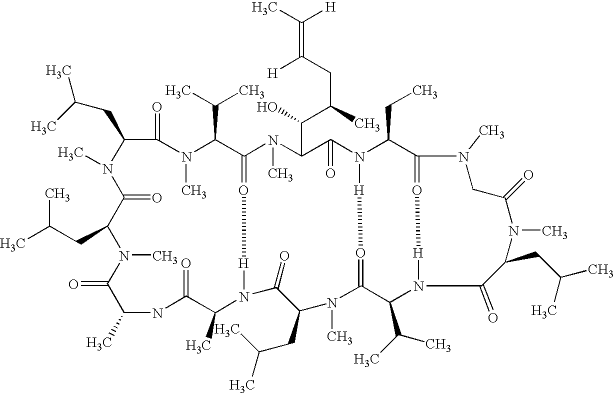

Each 6 mL sterile single-use glass vial, with a latex-free rubber stopper, contains a minimum fill of 5.0 mL of formulation. This fill contains a sufficient amount of cyclosporine, USP (325 mg) in propylene glycol, USP (5.2 mL) to deliver 300 mg in 4.8 mL.

When administered by oral inhalation, between 5.4 and 11.2% of the dose is directly delivered to the lung bronchiolar epithelium. As such, cyclosporine is locally available at the site of rejection and provides local immunosuppression that is achieved with lower systemic levels of exposure than are seen with either the oral or i.v. routes of administration. Therefore, the potential for systemic adverse events is reduced.

A formulation such as PULMINIQ™ may be administered as described in the table below. In one of many potential therapeutic regimens, when the maintenance dose has been achieved, 300 mg. or the maximum tolerated dose may be administered three times per week (e.g., Monday, Wednesday, and Friday) for at least two years.

Preferably, administration of the formulation such as PULMINIQ™ is initiated no later than 42 days after transplantation.

A typical schedule for dose titration follows:

| | | PULMINIQ ™ | Volume |
|---|---|---|---|
| | | Dose (mg) | (mL) |
| Titration | Day 1 | 100 mg | 1.5 |
| | Day 2 | 200 mg* | 3.0 |
| | Day 3-10 | 300 mg* | 4.8 |
| Maintenance (after 10 days) | 3 times weekly | 300 mg* | 4.8 |

*if tolerated

Dosing times may vary depending on the amount of formulation such as in the following chart:

| Dose | Dose Volume | Approximate |
|---|---|---|
| (mg) | (mL) | Dosing Time (min) |
| 100 mg | 1.5 | 15 |
| 200 mg | 3.0 | 30 |
| 300 mg | 4.8 | 45 |

Experimental evidence suggests that the effectiveness of cyclosporine is due to specific and reversible inhibition of immunocompetent lymphocytes in the G₀- or G₁-phase of the cell cycle. T-lymphocytes are preferentially inhibited. The T-helper cell is the main target, although the T-suppressor cells may also be suppressed. Cyclosporine also inhibits lymphokine production and release including interleukin-2 and T-cell growth factor (TCGF)(1).

### Uses of Aerosolized Cyclosporine

The invention is also directed to methods of preventing graft rejection in an organ transplant patient such as a lung transplant patient using the systems described herein.

In addition, the system may be used in a method to treat a pulmonary disorder or an immune disorder.

In a preferred embodiment, the present system is used to deliver aerosolized cyclosporine, which is used as a means for inhibiting the onset of graft rejection, preferably in lung transplant patients and heart/lung transplant patients. In one approach, the aerosolized cyclosporine is administered to a transplant recipient directly following transplantation. In this embodiment of the invention, the initial maximum dose of aerosolized cyclosporine is usually administered to the transplant recipient within 10 days following transplantation or prior to the development of any of the symptoms generally associated with lung transplant rejection.

In addition, the methods and systems of the invention may be used to prevent rejection in organ transplant recipients and for treatment of immune disorders. Such organ transplants include, but are not limited to, transplants of the lung, heart, liver, kidney, and bone marrow. An effective amount of an aerosolized cyclosporine formulation is administered, which means an amount sufficient to prevent development of an immune response that would lead to graft rejection in a transplant recipient.

The invention relies on the administration of aerosolized cyclosporine, typically cyclosporine A, using a system comprising an exhalation filter, trap or other means to minimize or prevent the escape of cyclosporine particles into the environment. The cyclosporine formulation may be provided in liquid form (as a solution), encapsulated form, attached to a carrier molecule or other carrier material, via a liposome, ie, suspension of cyclosporine within the membrane of a liposome, or as a dry powder or emulsion.

Liquid formulations of cyclosporine can comprise any recognized physiologically acceptable carrier or solvent for use in delivery of aerosolized formulations. Such carriers or solvents include but are not limited to ethanol, propylene glycol, polyethylene glycol, ethanol-propylene combinations, phospholipids, lipids, tetrahydrofurfuryl alcohol, polyethyleneglycol ether, glycerin and the like. Cyclosporine is soluble in lipids and organic solvents, having a solubility of about 80 mg/ml in alcohol at 25° C.

The amount of cyclosporine nebulized is typically from about 100 to 500 mg, more typically from 20 to 400 mg or 50 to 300 mg of aerosolized cyclosporine. A standard therapeutic dose of cyclosporine is 300 mg. The amount of cyclosporine delivered to the lung is typically between 5 and 50 mg.

In addition, the system may be used to treat subjects having T-cell mediated immune disorders such as type IV cell mediated (delayed-type) hypersensitivity, or autoimmune disorders. Autoimmune disorders which may be treated using aerosolized cyclosporine include, for example, systemic lupus erythematosus, myasthenia gravis, Grave's disease, Hashimoto's thyroiditis, rheumatoid arthritis, scleroderma, and pernicious anemia. An effective amount of the formulation refers to an amount sufficient to inhibit the immune response associated with the immune disorder.

Pulmonary disorders for which the system may use to treat may be inflammatory pulmonary disorders wherein the symptoms of the disease result from a local immune reaction in the lungs. Examples of such disorders include, but are not limited to, asthma, sarcoidosis, emphysema, cystic fibrosis, idiopathic pulmonary fibrosis, chronic bronchitis, allergic rhinitis and allergic diseases of the lung such as hypersensitivity pneumonitis and eosinophilic pneumonia. An effective amount of the formulation used in the system of the invention refers to an amount of cyclosporine sufficient to inhibit an immune response in the lung of a subject suffering from a pulmonary disorder, thereby decreasing the inflammation associated with the disorder.

In general, the total dose range of aerosolized cyclosporine should be sufficient to achieve concentration levels ranging between 5 mg and 30 mg in the lung, while most preferably a dose range sufficient to achieve concentration levels ranging between 5 mg and 15 mg in the lung is desirable. For example, a dose of between 20-400 mg of an aerosolized cyclosporine is administered, while most preferably a dose of aerosolized cyclosporine of between 50-300 mg is administered. Overall, doses of aerosolized cyclosporine may vary depending on the type and extent of lung disease; however, it is believed that doses needed to achieve a beneficial response will be less than the doses of aerosolized cyclosporine required to ameliorate transplant related inflammation. It may be necessary to use dosages outside these ranges in some cases, as will be apparent to those of ordinary skill in the art.

In certain instances, it may be desirable to co-administer to a subject, for example, exhibiting pulmonary disorder symptoms, aerosolized cyclosporine in conjunction with an additional agent/s. These agents may be administered by oral, parenteral or inhalation routes. Such agents include, for example, antibiotics, antivirals, immunosuppressives, or anti-inflammatory agents. Anti-inflammatory drugs include, for example, inhaled steroids 4×220 mgs/puff/day, prednisone 20-60 mg day, methotrexate 5-15 mg/week, azathioprine 50-200 mg/day. Determination of effective amounts is well within the capability of those skilled in the art.

The following examples are offered to illustrate but not to limit the invention.

### Example 1

PULMINIQ™ (NIF027) Cyclosporine Solution for Inhalation (CSI) (Lot Number Y127 0703, Novartis Pharma AG, Basel, Switzerland) was used. Each vial contained 5.2 mL of a 62.5 mg/mL solution of cyclosporine A in propylene glycol.

The aerosol system utilized the Aerotech™ II nebulizer (Lot Number 1664121, CIS-US, Inc., Bedford, Mass.) and the DeVilbiss® Model 8650D (Sunrise Medical, Somerset, Pa.) compressor set to 40 PSI to generate the CSI aerosol. A new nebulizer was used for each experimental run.

Listed in Table 1 are the respiratory filters that were tested. The trap filter used is the Conserve™ 50 Breathing Circuit Filters (Lot Number 322301, Pall Corporation, East Hills, N.Y.).

TABLE 1
Filters for Study CIPT-5120

| Filter | Manufacturer |
|---|---|
| Standard Aerotech ™ II Filter | CIS-US Inc., Bedford, MA |
| Lot Number 1664121 | |
| Iso-Gard ® HEPA Light | Hudson RCI, Upplands Väsby, |
| (Catalogue Number 28022) | Sweden |
| Lot Number 113924 | |

The breathing simulator was a Respirator Pump (Harvard Apparatus, Inc., Holliston, Mass.) producing a sine wave breathing pattern. The following settings were used for all experiments.
Inspiratory Rate=15 breaths/min
Inspiration=50%
cc per stroke=Vₜ (Tidal Volume)=500 mL

The filter resistance was measured for each test filter before and after the filter challenge. The filter was connected to the inlet/outlet port of a Hans Rudolph Series 1101 Breathing Simulator (Kansas City, Mo.). The “Normal” breathing default configuration was used. The filter was allowed to run for several cycles to stabilize and the “Peak Airway Pressure (cmH₂O)” and “Peak Inhale Flow (LPM)” was recorded. Resistance is expressed by the following formula.

$$Resistance ( cm {H}_{2} {O}^{0.5} \cdot min / L ) = \frac{{[ Peak Airway Pressure ( cm {H}_{2} O ) ]}^{0.5}}{[ Peak Inhale Flow ( L / min ) ]} \qquad$$

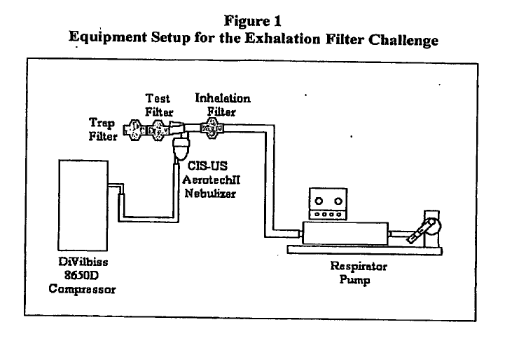

The filter challenge comprised the following procedure: One 6 mL vial of PULMINIQ™ (NIF027) was emptied into a new Aerotech™ II nebulizer and the equipment set according to FIG. 1 with a test filter. The respirator pump and 8650D compressor (set at 40 PSI) was turned on. The nebulizer was allowed to run dry and the compressor and pump were turned off. The test filter was removed and then connected to the inlet/outlet port of a Hans Rudolph Series 1101 Breathing Simulator and the resistance measured.

The trap filter was extracted with ethanol (30 mL) followed by positive pressure to assist with draining the filter. Four more extractions were performed for a total of five extractions. The extracts were transferred into a 200 mL volumetric flask and Q.S. to the mark with additional ethanol. Three replicates were performed for each test filter type from Table 1.

The trap filter samples were analyzed. Particularly, a 20 mL aliquot from each individual sample was analyzed for total mass of cyclosporine.

A summary of the study results are listed in Tables 2 and 3. The average volume dispensed from one vial of NIF027 Cyclosporine Solution for Inhalation was 4.9 mL.

TABLE 2
Filter Challenge Results
(Average ± S.D.)

| | CsA Captured on | Escaped | Filter |
|---|---|---|---|
| | Trap Filter | CsA* | Efficiency** |
| Filter | (mg) | (%) | (%) |
| Standard Aerotech ™ | 55.70 ± 3.33 | 18.33 ± 1.07 | 57.89 ± 2.52 |
| II Filter | | | |
| Iso-Gard ® HEPA | 0.35 ± 0.02 | 0.12 ± 0.00 | 99.73 ± 0.01 |
| Light | | | |

*Calculation based on total of CsA placed into the nebulizer.
**Calculation based on CsA recovered on exhalation filter, in vitro Delivered Dose of NIF027 Cyclosporine Solution for Inhalation.

TABLE 3
Filter Resistance and Dose Time Results
(Average ± S.D.)

| | Filter Resistance | | Dose |
|---|---|---|---|
| | (cmH₂O^{0.5} · min/L) | | Time |
| Filter | New | End | (Minutes) |
| Standard Aerotech ™ | 0.0190 ± 0.0008 | 0.0268 ± 0.0018 | 39.9 ± 4.2 |
| II Filter | | | |
| Iso-Gard ® HEPA | 0.0234 ± 0.0006 | 0.0489 ± 0.0024 | 38.9 ± 1.6 |
| Light | | | |

The results demonstrate that the Iso-Gard® HEPA Light (Hudson RCI) is a suitable filter to be used with the Aerotech™ II nebulizer during NIF027 Cyclosporine Solution for Inhalation dosing. The filter prevents 99.73% of the exhaled aerosol from escaping to the local environment. In addition, the Iso-Gard® HEPA Light filter flow resistance was negligible for human subjects.

### Example 2

Similarly, the Conserve™ 50 Breathing Circuit Filter (Pall Corporation) was tested using a similar procedure. This pleated respiratory filter contains hydrophobic resin bonded inorganic fibers. The Conserve™ 50 Breathing Circuit Filter exhibited sufficient filtering, but the filter flow resistance was not measured, as the filter had clogged. These results indicate that the Conserve™ 50 Breathing Circuit Filter is not suitable for use with the cyclosporine solution because it did not maintain low filter flow resistance.

1. An apparatus for administration of aerosolized cyclosporine, comprising,
a pressurized delivery device coupled to an exhalation filter or trap, wherein the exhalation filter or trap is capable of maintaining an efficiency of greater than 90% during usage with a liquid formulation comprising cyclosporine for aerosolization by the device.

2. The apparatus according to claim 1, wherein said exhalation filter is capable of maintaining an increase in filter resistance between a new filter and after usage of the formulation of less than 0.1 cmH₂O^{0.5}·min/L.

3. The apparatus according to claim 2, wherein the exhalation filter is a hydrophobic high efficiency particulate air (HEPA) filter.

4. The apparatus according to claim 3, wherein the HEPA filter comprises a polypropylene and/or acrylic medium.

5. The apparatus according to claim 3, wherein the exhalation filter is an Isogard HEPA Light filter.

6. The apparatus according to claim 1, wherein said trap comprises a solvent with a high affinity for cyclosporine.

7. The apparatus according to claim 6, wherein said solvent trap captures escaping cyclosporine particles.

8. A system comprising the apparatus of claim 1 and a liquid formulation comprising cyclosporine for aerosolization by the device.

9. The system according to claim 8, wherein the exhalation filter is a hydrophobic high efficiency particulate air (HEPA) filter.

10. The system according to claim 9, wherein the HEPA filter comprises a polypropylene and/or acrylic medium.

11. The system according to claim 9, wherein the exhalation filter is an Isogard HEPA Light filter.

12. The system according to claim 8, wherein said liquid formulation comprises cyclosporine and a solvent selected from the group consisting of ethanol, propylene glycol, polyethylene glycol, ethanol-propylene combinations, phospholipids, lipids, tetrahydrofurfuryl alcohol, polyethyleneglycol ether and glycerin.

13. The system according to claim 8, wherein said liquid formulation comprises cyclosporine and propylene glycol.

14. The system according to claim 8, wherein said liquid formulation comprises cyclosporine and ethanol.

15. The system according to claim 8, wherein the pressurized delivery device is a nebulizer and the formulation is present in an amount less than 10 mL.

16. The system according to claim 15, wherein the formulation is present in an amount less than 7 mL.

17. The system according to claim 15, wherein the formulation is present in an amount less than 5 mL.

18. The system according to claim 15, wherein the formulation is present in an amount less than 3 mL.

19. The system according to claim 15, wherein the formulation is present in an amount less than 2 mL.

20. The system according to claim 12, wherein the formulation contains 62.5 mg/ml of cyclosporine A.

21. The system according to claim 16, wherein the formulation contains 325 mg cyclosporine and 5.2 ml propylene glycol.

22. A method of treating a pulmonary disorder comprising: administering aerosolized cyclosporine using the system according to claim 6, to a lung of a subject having a pulmonary disorder.

23. The method according to claim 22, wherein the pulmonary disorder is cystic fibrosis.

24. The method according to claim 22, wherein the lung is a transplanted lung.

25. A method of preventing graft rejection in an organ transplant patient comprising administering to a subject an effective dose of aerosolized cyclosporine using the system according to claim 8.

26. The method according to claim 25, wherein the organ is a lung.

27. The method as according to claim 26, wherein the formulation is administered directly after lung transplantation.

28. A method for selecting an exhalation filter for use in a system comprising a nebulizer coupled to the exhalation filter, wherein the nebulizer contains a liquid formulation comprising cyclosporine, comprising:
turning on a compressor and a respirator pump in a breathing apparatus to nebulize the solution, wherein the apparatus comprises a test exhalation filter, the compressor, the respirator pump, an inhalation filter, a trap filter, and a nebulizer containing said liquid cyclosporine formulation wherein the components of the apparatus are coupled to each other to form the breathing apparatus capable of simulating breathing;
turning off the compressor and the respirator pump after the nebulizer runs dry;
measuring the test filter efficiency by testing the trap filter to measure the quantity of cyclosporine that passed through the test filter;
measuring the filter resistance of the test filter, wherein a filter efficiency of greater than 90%, and an increase in filter resistance between the test filter before the turning on step and after the turning off step of less than 0.1 cmH₂O^{0.5}·min/L, are indicative of an appropriate exhalation filter to be used with said solution.
